(19) United States
(12) Patent Application Publication
(10) Pub. No.: US 2014/0140887 A1
(43) Pub. Date: May 22, 2014

(54) ROOM DISINFECTION METHOD

(71) Applicant: Jamaal Parham, Jonesboro, GA (US)

(72) Inventor: Jamaal Parham, Jonesboro, GA (US)

(21) Appl. No.: 13/682,032

(22) Filed: Nov. 20, 2012

(51) Int. Cl. A61L 2/18

(52) U.S. Cl. CPC A61L 2/18
USPC 422/4

(57) ABSTRACT

A system for disinfecting a room comprising a spray dispenser which dispenses a solution having antimicrobial properties at predetermined time intervals so as to provide continuous disinfection of the room, in combination with an HVAC system having an air filter which has been treated with a solution also having antimicrobial properties so as to further aid in disinfection of air entering the room. The system is designed to provide around-the-clock disinfection of the room thereby reducing the reliance on personnel to disinfect a room, especially in a healthcare setting such as in an emergency room or a surgical theatre.

### BACKGROUND OF THE INVENTION

1. Field of the Invention

The present invention relates to a comprehensive method for disinfecting and sanitizing a room by dispensing an antimicrobial solution into the room and filtering air entering the room with a filter that has also been treated with an antimicrobial solution.

2. Description of Related Art

The present invention is primarily directed to the healthcare setting where the presence of microorganisms can be a matter of life or death. Healthcare settings encompass a variety of different facilities including hospitals, nursing homes, residential treatment facilities, emergency rooms, surgery theatres, dental offices, blood banks, dialysis centers, and many others. In each of these environments, there is a need to rid the environment of harmful microorganisms.

Healthcare-associated infections (HAIs) are infections caused by a wide variety of common and unusual bacteria, fungi, and viruses during the course of receiving medical care. These include escherichia coli, staphylococcus aureus, klebsiella pneumoniae, penicillium pinophilum, chaetomium globosum, and bacillus subtilis, for example. While the advances in medical care and treatment cannot be denied, many of those advances come with a risk of HAIs. Wherever patient care is provided, adherence to infection prevention guidelines is needed to ensure that all care is provided in a safe environment. This includes traditional hospital settings as well as outpatient surgery centers, long-term care facilities, rehabilitation centers, and community clinics. The prevention and control of viruses and bacterial infections requires measures to prevent their transmission and the appropriate use of antimicrobial agents. Antiseptics and disinfectants are widely used in healthcare settings and in various other industries to prevent the spread of bacteria and viruses. Their use is regulated by several governmental agencies such as the EPA, CDC and OSHA. In addition to their use in institutional and industrial settings, these products are also becoming increasingly popular with household consumers as a result of a heighted awareness of the dangers associated with microbials. It has been estimated that the global market for antiseptics and disinfectants in 2010 was approximately $3.8 billion. The market is expected to reach $7.1 billion by 2016. The U.S. is the world leader in the antiseptics and disinfectants market constituting about 41% of the total antiseptics and disinfectants market in 2010. The U.S. market is expected to increase to nearly $2.9 billion by 2016. While development of new antimicrobial agents is the cornerstones of activities to protect our health, successful prevention can most readily be obtained by effective use of existing antimicrobial agents thereby reducing the population of microorganisms in the environment.

Many prior attempts have been made to address the ills associated with microorganisms in the healthcare setting. Some of the prior art methods of ridding these environments of airborne and surface laden microorganisms include the use of spray guns, foggers, fumigators, and the like.

One prior art technique for disinfection of a room is taught by U.S. Patent Application Publication No. 2006/0078461 which discloses a method and apparatus for disinfecting an entire room in one operation that includes providing a sufficiently large container having a liquid disinfectant and aerosol, and a manually actuated valve and nozzle that includes a spray pattern for a uniform distribution of aerosol spray. To use the device, the aerosol container is placed at a central location in the room based on its distribution pattern and, once activated, the room is kept closed so that the entire disinfectant contents can be continuously dispersed for total disinfection of the room in a single operation until the contents of the container has been emptied. However, like many other prior art techniques for disinfecting a room, this approach does not effectively account for bacteria or germs subsequently entering the room via the HVAC system for the room.

HVAC systems can be a significant source of airborne bacteria and viruses entering a room even in the presence of an existing room disinfectant system. In such instances, new microorganism can enter the room as soon as the disinfectant treating the room is depleted. What is needed is a system for decontaminating a room which is effective in maintaining the disinfected status of the room even in the presence of an HVAC system which may supply airborne bacteria and viruses to the room.

### BRIEF SUMMARY OF THE INVENTION

Accordingly, one object of the invention is to provide a room disinfections system which can effectively disinfect the air and surfaces in a room or other enclosed space by eliminating or significantly reducing bacteria, viruses, and other germs in the room.

It is another object of the present invention to provide a room disinfecting and sterilizing system which is costs effective and easy to use.

It is yet another object of the present invention to provide a room disinfecting and sterilizing system which significantly reduces the reliance on personnel to decontaminate a room.

It is another object of the present invention to provide a room disinfection system which disinfects a room while healthcare personnel remain in the room to provide their normal healthcare services.

It is still another object of the present invention to provide a room decontamination system which effectively eliminates microorganisms in a room as well as eliminate airborne microorganisms entering the room via an HVAC system.

The above objects are achieved by a system for disinfecting and sterilizing a room comprising a metered spray or mist dispenser which dispenses a solution having antimicrobial properties at predetermined time intervals so as to provide continuous disinfection of the room, in combination with an HVAC system having an air filter which has also been treated with an oil-based, antimicrobial solution so as to further aid in disinfection of air in the room. The system is designed to provide continuous decontamination of the room. The metered spray or mist dispenser may be a pressurized container such as an aerosol canister and any other suitable container such as a fogger or a sprayer device. Thus, around-the-clock treatment of the room is obtained, thereby reducing he reliance on personnel to disinfect a room such an emergency room or a surgical theatre.

### DETAILED DESCRIPTION OF THE INVENTION

Before describing the present invention in detail, it is to be understood that this invention is not limited to particularly exemplified systems which may, of course, vary. It is also to be understood that the terminology used herein is for the purpose of describing particular embodiments of the invention only, and is not intended to limit the scope of the invention in any manner. All patents and patent applications cited herein, whether supra or infra, are hereby incorporated by reference in their entirety to the same extent as if each individual patent or patent application was specifically and individually indicated to be incorporated by reference. Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

According to a first aspect of the present invention there is provided a method of simultaneously disinfecting air and surfaces in a room as well as and filtering and disinfecting air entering the room. The method includes the steps of selecting a spray dispenser. In the preferred embodiment of the present invention, such a spray dispenser will be selected from the group consisting of an aerosol spray dispenser, a fogger, and a pump spray dispenser. Preferably, such spray dispenser will be an aerosol spray dispenser with means for dispensing a spray at predetermined time intervals and for predetermined durations. In this regard, the dispenser may be provided with a programmable microprocessor and a power supply for spraying an antimicrobial mist at preprogrammed time intervals.

The method further requires selecting a fluid disinfecting agent and, thereafter, placing a predetermined amount, by volume, of such fluid disinfecting agent selected into the spray dispenser selected. In the preferred embodiment of the invention, such fluid disinfecting agent is a hospital-grade, broad spectrum disinfectants registered with the EPA. Preferably, such fluid disinfecting agent is Clorox® Broad Spectrum Quaternary Disinfectant Cleaner available from Clorox Professional Products Company. It will be clear to those skilled in the art that various other disinfectant agents may be selected and used based upon the particular microorganism or biological agent at issue. In this regard, the present invention may be adapted for use as a countermeasure against biological agents commonly associated with chemical and biological warfare.

In accordance with another aspect of the present invention, the disinfecting agent is dispersed at predetermined time intervals. It is preferred that such predetermined time interval be between about 5 minutes to about 60 minutes and the spray duration be between about 5 seconds and 60 seconds. Preferably, the predetermined time interval will be between about 20 minutes to about 40 minutes and the spray duration will be between about 10 seconds and 30 seconds. Thus, the disinfecting agent is dispersed into the room and air and surfaces in the room are disinfected due, in part, to the presence of the disinfectant.

The method of the present invention also includes the step of providing a filtration means for filtering air entering the room and then placing a predetermined amount of an oil-based disinfectant on said filtration means so that air entering the room passes through said filtration means, further reducing the amount of bacteria, viruses and other airborne germs which might otherwise enter the room. In the preferred embodiment of the invention, such oil-based disinfectant is selected from the group consisting of pine-oil based disinfectants and thyme-oil based compositions having antimicrobial properties. Preferably, such fluid disinfecting agent selected is Pine-Sol® cleaner available from The Clorox Company. Pine Sol® is a pine oil based product that also contains isopropyl alcohol and quaternary ammonium compounds. The filter is preferably a HEPA (High Efficiency Particulate Air) filter. To qualify as HEPA by US government standards, an air filter must remove 99.97% of all particles greater than 0.3 microns from the air that passes through. Application of the antimicrobial agent to the filter may be accomplished either before, during, or after manufacture of the filter. For example, the filter medium may be immerged in the antimicrobial solution prior to assembly of the filter. In the alternative, the antimicrobial solution may be applied by spraying the solution onto the filter medium subsequent to manufacture of the filter. In another embodiment of the present invention, the antimicrobial disinfectant agent is in the form of a gel which may be spread onto the filter medium. It will be clear to those skilled in the art that although an oil-based disinfectant is described in connection with the preferred embodiment of the present invention, other disinfectants such as an alcohol-based disinfectant solution or gels may be used without departing from the scope and spirit of the present invention.

Optionally, either the spray dispenser of the present invention, the air filter of the present invention, or both, may be supplemented by adding a deodorizing agent. In the presently preferred embodiment of the invention, such deodorizing agent is selected from the group consisting of Febreze and Lysol although it will be clear to those skilled in the art that many other deodorizer, scents and fragrances may be used without departing from the scope and spirit of the present invention. It is most preferred that such deodorizing agent is Febreze.

When the spray dispenser of the present invention also includes a deodorizing agent, the predetermined amount, by volume, of the disinfecting agent will generally be between about 75 to about 99 percent by volume. It is most preferred that such predetermined amount, by volume, of such fluid disinfecting agent be between about 85 to about 95 percent by volume. Accordingly, the predetermined amount, by volume, of the deodorizing agent will be between about 1 to about 25 percent by volume. Preferably, such predetermined amount, by volume, of the deodorizing agent is between about 5 to about 15 percent by volume.

What has been described is a comprehensive, dual-approach room disinfection system that automatically disinfects a room such as a hospital surgical theatre thereby reducing or potentially eliminating the need to use high-cost personnel to disinfect such an environment of harmful bacteria, viruses, germs and other microorganisms. While a presently preferred and various alternative embodiments of the present invention have been described in sufficient detail above to enable a person skilled in the relevant art to make and use the same, it should be obvious that various other adaptations and modifications can be envisioned by those persons skilled in the relevant art without departing from scope and spirit of the invention as embodied in the appended claims.

I claim:

1. A method for disinfecting air and surfaces in a room having an HVAC system, comprising the steps of:
(a) providing a spray dispenser comprising a first antimicrobial solution;
(b) dispensing said first antimicrobial solution from said spray dispenser into the room;
(c) providing a filter for the HVAC system, said filter being treated with a second antimicrobial solution; and
(d) operating the HVAC system to blow filtered air into the room whereby air and surfaces in the room are disinfected.

2. The method of claim 1, wherein said spray dispenser is selected from the group consisting of an aerosol spray dispenser, a fogger, and a pump spray dispenser.

3. The method of claim 2, wherein said spray dispenser is an aerosol spray dispenser.

4. The method of claim 1, wherein said spray dispenser further comprises means for dispensing a spray at predetermined time intervals and for predetermined intervals.

5. The method of claim 1, wherein said first antimicrobial solution is a hospital-grade, broad spectrum disinfectants registered with the EPA.

6. The method of claim 1, wherein said second antimicrobial solution is selected from the group consisting of pine-oil based disinfectants and thyme-oil based disinfectants.

7. The method of claim 1, wherein said filter further comprises a high efficiency particulate air filter.

8. The method of claim 6, wherein said second antimicrobial solution is applied to said filter subsequent to manufacture of said filter.

9. The method of claim 1, further including the step of providing a deodorizing agent.

10. A method for disinfecting air and surfaces in a room having an HVAC system, comprising the steps of:
(a) providing an aerosol spray dispenser comprising Clorox Broad Spectrum Quaternary Disinfectant Cleaner as a first antimicrobial solution;
(b) dispensing said first antimicrobial solution from said aerosol spray dispenser into the room;
(c) providing a HEPA filter for the HVAC system, said HEPA filter being treated with Pine-Sol a second antimicrobial solution; and
(d) operating the HVAC system to blow filtered air into the room whereby air and surfaces in the room are disinfected.

11. The method of claim 10, wherein said aerosol spray dispenser further comprises a programmable microprocessor which is programmable to dispense a spray at predetermined time intervals and for predetermined durations.

12. The method of claim 11, wherein said predetermined time interval is between 5 minutes and 60 minutes and said spray duration is between 5 seconds and 60 seconds.
